(19) United States
(12) Patent Application Publication
(10) Pub. No.: US 2022/0152236 A1
(43) Pub. Date: May 19, 2022

(54) Multi-Band Germicidal Irradiation Apparatus

(71) Applicant: Aleddra Inc., Renton, WA (US)

(72) Inventors: Chia-Yiu Maa, Bellevue, WA (US); Chun-Te Yu, Bellevue, WA (US)

(21) Appl. No.: 17/140,673

(22) Filed: Jan. 4, 2021

Related U.S. Application Data
(63) Continuation-in-part of application No. 17/137,763, filed on Dec. 30, 2020.
Application No. 17/137,763 is a continuation-in-part of application No. 17/099,271, filed on Nov. 16, 2020.

(51) Int. Cl. A61L 2/10; A61L 2/26; A61L 2/08

(52) U.S. Cl. CPC A61L 2/10; A61L 2/26; A61L 2202/14; A61L 2202/11; A61L 2/084

(57) ABSTRACT

A multi-band germicidal irradiation apparatus includes a radiation source, a driver, and an optical filter. The radiation source emits wavelengths in three wavelength bands, 190˜230 nm, 230˜315 nm, and 315˜700 nm. The optical filter is a band-stop filter that filters the wavelength in a wavelength range of 240˜315 nm and permits the wavelength in wavelength ranges of 190˜230 nm and 315˜700 nm to pass through. The radiation source may include an excimer lamp having a gas or mixture of gases, or it may include one or more light emitting diodes (LEDs). When the apparatus further includes a controller, the controller is configured to limit a total dispensed ultraviolet (UV) dosage (TDD) emitted by the apparatus over a predefined period not exceeding an UV threshold limit value (TLV) dosage defined by American Conference of Governmental Industrial Hygienists (ACGIH).

### BACKGROUND

### Technical Field

The present disclosure is part of a Continuation-in-Part (CIP) of U.S. patent application Ser. No. 17/137,763, filed 30 Dec. 2020, which is a CIP of U.S. patent application Ser. No. 17/099,271, filed 16 Nov. 2020, the contents of which being incorporated by reference in their entirety.

### Description pf Related Art

Germicidal irradiation refers to the use of a radiant source emitting primarily ultraviolet (UV) wavelength in a range of 190 nm˜400 nm for disinfecting against bacteria and viruses in the air or on a surface. Germicidal irradiation applications are not new. There is, however, a renewed interest of the germicidal irradiation technologies and applications due to the COVID-19 pandemic. It is shown that an ultraviolet-C (UVC) dosage of 5 mJ/cm² can disinfect against the SARS-CoV-II virus (the COVID-19 virus) with a 99.99% kill rate. This gives rise to the proliferation of germicidal irradiation devices on the market, and so are the many UVC burn incidents on skin and/or eyes due to UVC over-exposure.

Recent studies have demonstrated that a far-UVC radiant source emitting a wavelength in a wavelength range of 190˜230 nm has the effect of killing bacteria and viruses, yet without the side effect of causing skin and eye damages to a user. One such study can be found at https://www.cuimc.columbia.edu/news/far-uvc-light-safely-kills-airborne-coronaviruses. This leads to the possibility of using a far-UVC radian source with a 190˜230 nm wavelength range in a germicidal irradiation equipment. However, a radiant source capable of emitting a wavelength in a wavelength range of 190˜230 nm may also emit a harmful wavelength in the regular UVC wavelength range of 230˜280 nm and/or the UVB wavelength range of 280˜315 nm. In U.S. Pat. No. 10,071,262, Randers-Pehrson et al. teaches the use of an excimer lamp having a peak wavelength in a wavelength range of 190˜230 nm and a band-pass filter for filtering all UV wavelengths in the 230˜400 nm range and permitting a visible light to pass through.

It is agreeable that harmful UVC wavelengths in the 230˜280 nm range and the UVB wavelengths in the 280˜300 nm range should be removed. American Conference of Governmental Industrial Hygienists (ACGIH) has published a UV Safety Guidelines as shown in FIG. 1 (ACGIH ISBN: 0-9367-12-99-6). It is evident that the UV Threshold Limit Values (TLVs) is the lowest for the wavelengths in the 230˜300 nm range, where the TLV is the maximum allowable dosage (in mJ/cm²) for each wavelength over an eight-hour period. However, there are two important observations from the ACGIH UV Safety Guidelines. Firstly, the tolerance level for UVA wavelengths in the 315˜400 nm range is remarkably high, greater than 10³ mJ/cm². This means it would be safe to administer some UVA to an occupant without any health risk. Moreover, it is known UVA wavelengths could damage the cellular wall of bacteria thus inhibiting bacteria reproduction. Therefore, it would be beneficial not to filter the UVA wavelengths and to use its bacteria inhibition feature. Secondly, it can be seen from FIG. 1, the TLV for the wavelengths in the 190˜230 nm range is not without restriction. For example, for a 222 nm wavelength, the TVL is set to be 22 mJ/cm². Therefore, it is important to limit the UV dosage emitted by a far-UVC radiant source so as not to violating the ACGIH UV Safety Guidelines or causing any harm to an occupant.

The present disclosure proposes a germicidal irradiation apparatus that uses a radiant source for generating UV wavelengths and a band-pass filter for filtering the wavelength in a wavelength range of 240˜315 nm. Additionally, when further comprising a controller, the controller is configured to limit a total dispensed UV dosage (TDD) emitted by the apparatus over a predefined period not exceeding a TLV dosage defined ACGIH.

### SUMMARY

In one aspect, the multi-band germicidal irradiation apparatus comprises a radiant source, a driver, and an optical filter. The driver is configured to convert an external power to an internal power to activate the radiation source. The radiation source is configured to generate a first wavelength in a wavelength range of 190˜230 nm, a second wavelength in a wavelength range of 315˜700 nm. The optical filter is configured to substantially reduce (e.g., over 50%) the radiation of the radiation source in a wavelength range of 240˜315 nm. Moreover, the optical filter is configured to transmit at least a part of the radiation of the radiation source in wavelength ranges of 190˜230 nm and 315˜700 nm. The optical filter is a band-stop filter that would reduce substantially the penetration of the wavelengths in the 240˜315 nm range. The optical filter may reduce the penetration of the wavelengths in the 230˜240 nm range, though not significantly. With this band-stop filter, the UVA wavelengths in the 315˜400 nm range are permitted to pass through, thus yielding the germicidal irradiation benefit of the UVA wavelength in inhibiting bacteria reproduction.

There are different choices for the radiant source. In some embodiments, the radiation source includes an excimer lamp having a gas or a combination of a plurality of gases for producing a first wavelength in the wavelength range of 190˜230 nm, a second wavelength in the wavelength range of 315˜700 nm.

It is known that through an electric discharge, krypton-chloride (KrCl) can generate 222 nm wavelength, krypton-bromine (KrBr) can generate 207 nm wavelength, argon-fluorine (ArF) can generate 193 nm wavelength, and krypton-iodine (KrI) can generate 190-191 nm wavelength. Therefore, in some embodiments, the excimer lamp comprises krypton-chloride (KrCl), krypton-bromine (KrBr), argon-fluorine (ArF), krypton-iodine (KrI), or a combination thereof, configured to generate a first wavelength in the wavelength range of 190˜230 nm.

There are different choices for generating a UVA wavelength in 315˜400 nm range. It is known that through an electric discharge, iodine (12) can generate 342 nm wavelength, and xenon-fluorine (XeF) can generate 351 nm wavelength. Therefore, in some embodiments, the excimer lamp comprises iodine (I₂), xenon-fluorine (XeF), or a combination thereof, configured to generate a wavelength in the wavelength range of 315˜400 nm.

In some embodiments, the excimer lamp comprises a gas configured to generate a wavelength in the wavelength range of 400˜700 nm, this is so that when the excimer lamp is in operation, the irradiation is visible to an occupant.

A combination of a plurality of gases do not necessarily mean these gases are mixed together. These gases may or may not mixed together, depending on the embodiments. In fact, in some embodiments, the excimer lamp comprises two or more gases. Moreover, each gas is stored in a separate enclosed chamber and the gases are not mixed.

Excimer lamp is not the only choice for generating UV wavelengths. Light emitting diodes (LEDs) can emit almost any wavelength in the 190˜700 nm range. In some embodiments, the radiation source includes one or more LEDs configured to emit a first wavelength in the wavelength range of 190˜230 nm, a second wavelength in the wavelength range of 315˜700 nm.

It is foreseeable to use just one type of LEDs for generating the first, the second, and the third wavelengths. For better performance, however, it may be more reasonable to use multiple types of LEDs. In some embodiments, the radiation source includes more than one types of LED wherein each type of LED having a different spectral power distribution. For example, one type of LED emits wavelengths mainly in the 190˜230 nm range, and a second type of LED emits wavelengths mainly in the 315˜700 nm range.

In some embodiments, the apparatus further comprises a controller. The controller is connected to the driver and is configured to adjust a radiant power emitted by the radiant source. Moreover, the controller is configured to limit a total dispensed UV dosage (TDD) emitted by the apparatus over a predefined period not to exceed an UV threshold limit value (TLV) dosage defined by ACGIH. For example, for the 222 nm wavelength, the apparatus is configured to emit less than 22 mJ/cm² over an eight-hour period, as defined by ACGIH UV Safety Guidelines.

The radiant power is the radiant energy emitted by a radiant source and is measured in milli-Watts or mW. The irradiance is defined as the radiant energy per unit area, measured in mW/cm². The Germicidal Irradiation Dosage, or simply Dosage, can be defined as:

Dosage (mJ/cm²)=Irradiance (mW/cm²)×Time (second)

From this definition, the germicidal irradiation dosage depends on two factors: the irradiance and the time (of exposure under a given irradiance). To dispense a certain dosage, e.g., 5 mJ/cm², one can use a high-power radiant source (resulting in a higher irradiance) with a short exposure time or use a low-power radiant source (resulting in a lower irradiance) with a longer exposure time.

In some embodiments, the TDD is controlled by either or both of: (1) the controller adjusts an operating time of the radiant source during the predefined period (e.g., through the turning on or off the driver), and (2) the controller adjusts a radiant power emitted by the radiant source (e.g., through adjusting the driver output wattage).

In another aspect, the multi-band germicidal irradiation apparatus comprises a radiant source, a driver, a controller, and an optical filter. The driver is configured to convert an external power to an internal power to activate the radiation source. The controller is connected to the driver and is configured to adjust a radiant power emitted by the radiant source. The radiation source is configured to generate a first wavelength in a wavelength range of 190˜230 nm, a second wavelength in a wavelength range of 400˜700 nm. The optical filter is configured to substantially reduce (e.g., over 50%) the radiation of the radiation source in a wavelength range of 240˜400 nm. Moreover, the optical filter is configured to transmit at least a part of the radiation of the radiation source in wavelength ranges of 190˜230 nm and 400˜700 nm. The optical filter is a band-stop filter that would reduce substantially the penetration of the wavelengths in the 240˜400 nm range. The optical filter may reduce the penetration of the wavelengths in the 230˜240 nm range, though not significantly. With this band-stop filter, the UVA wavelengths in the 315˜400 nm range it are not permitted to pass through.

Sometimes it is preferable to operate a radiant source to deliver a UV dosage much lower than the ACGIH TLV, e.g., when someone is overly sensitive to UV exposure. Therefore, in some embodiments, the present disclosure supports a mild mode operation wherein the controller is configured to operate the radiant source such that the UV dosage received by the object is at least 25% below the UV TLV dosage defined by the ACGIH.

Sometimes it is preferable to operate a radiant source to deliver a UV dosage much higher than the ACGIH TLV, e.g., for buses, subways, and elevators. In these environments, occupants come and go thus making these environments a potential infection hotspot. It would be reasonable to accelerate the germicidal irradiation for these environments by increasing the UL dosages above ACGIH TLVs, and not to worry about occupants being over-exposed with UV since they would never stay in these environments for eight hours. Therefore, in some embodiments, the present disclosure supports a boost mode operation wherein the controller is configured to operate the radiant source such that the UV dosage received by the object is at least 25% above the UV TLV dosage defined by the ACGIH.

When an environment is not occupied, e.g., at night or during off hours, it would be reasonable to maximize the radiant power of a radiant source to have a deep sanitation of the environment over a short period of time such as 30 minutes or 60 minutes, or even longer. Therefore, in some embodiments, the present disclosure supports a full sanitation mode operation wherein the controller is configured to maximize a radiant power emitted by the radiant source over less than 4 hours.

It is foreseeable and in fact preferable that some embodiments of the disclosed apparatus would support the regular mode, the mild mode, the boost mode, and/or the full sanitation mode and allow a user or a scheduler to switch from one mode to another.

### BRIEF DESCRIPTION OF THE DRAWINGS

The accompanying drawings are included to aid further understanding of the present disclosure, and are incorporated in and constitute a part of the present disclosure. The drawings illustrate a select number of embodiments of the present disclosure and, together with the detailed description below, serve to explain the principles of the present disclosure. It is appreciable that the drawings are not necessarily to scale, as some components may be shown to be out of proportion to size in actual implementation in order to clearly illustrate the concept of the present disclosure.

FIG. 1 The Threshold Limit Values (dosage) according to ACGIH UV Safety Guidelines.

FIG. 2 schematically depicts a diagram of an embodiment of the present disclosure using an excimer lamp.

FIG. 3 shows the picture of an excimer lamp with 4 mini tubes.

FIG. 4 shows the spectral power distribution on an excimer lamp with KrCl and Kr₂Cl at 600 mbar.

FIG. 5 shows the transmittance curve of a band-stop filter.

FIG. 6 shows the spectral power distribution of the examiner lamp with KrCl and Kr₂Cl after being filtered by the band-stop filter.

FIG. 7 shows the spectral power distribution of another excimer lamp with KrCl and I₂ at 200 mbar.

FIG. 8 shows the spectral power distribution of the excimer lamp with KrCl and I₂ after being filtered by the band-stop filter.

FIG. 9 shows an operation schedule of the first embodiment of the present disclosure.

FIG. 10 schematically depicts a diagram of another embodiment of the present disclosure using an LED radiant source.

FIG. 11 shows the spectral power distribution on the LED radiance source with a peak wavelength at 215 nm.

FIG. 12 shows the transmittance curve of another band-stop filter.

FIG. 13 shows the spectral power distribution on the LED radiance source after being filtered by the second band-stop filter.

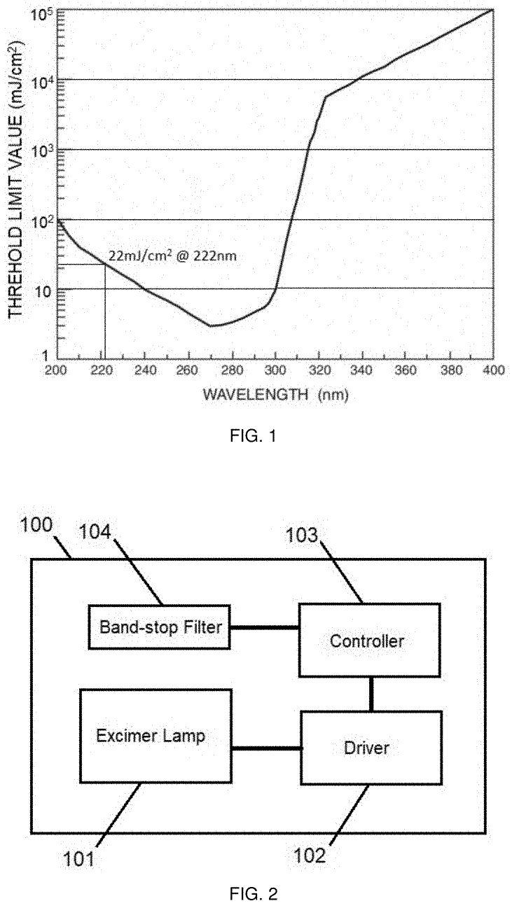

### DETAILED DESCRIPTION OF PREFERRED EMBODIMENTS

### Overview

Various implementations of the present disclosure and related inventive concepts are described below. It should be acknowledged, however, that the present disclosure is not limited to any particular manner of implementation, and that the various embodiments discussed explicitly herein are primarily for purposes of illustration. For example, the various concepts discussed herein may be suitably implemented in a variety of multi-band germicidal irradiation apparatuses having different form factors.

The present disclosure is a multi-band germicidal irradiation apparatus includes a radiation source, a driver, and an optical filter. The radiation source emits wavelengths in three wavelength bands, 190˜230 nm, 230˜315 nm, and 315˜700 nm. The optical filter is a band-stop filter that filters the wavelength in a wavelength range of 240˜315 nm and permits the wavelength in wavelength ranges of 190˜230 nm and 315˜700 nm to pass through. The radiation source may include an excimer lamp having a gas or mixture of gases, or it may include one or more light emitting diodes (LEDs). When the apparatus further includes a controller, the controller is configured to limit a total dispensed UV dosage (TDD) emitted by the apparatus over a predefined period not exceeding an UV TL dosage defined by ACGIH.

### EXAMPLE IMPLEMENTATIONS

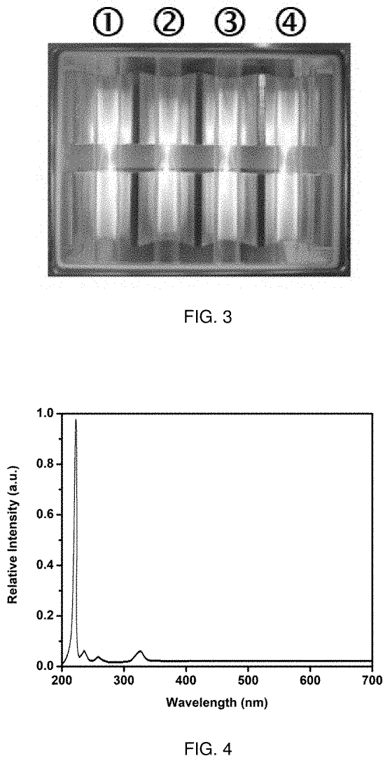

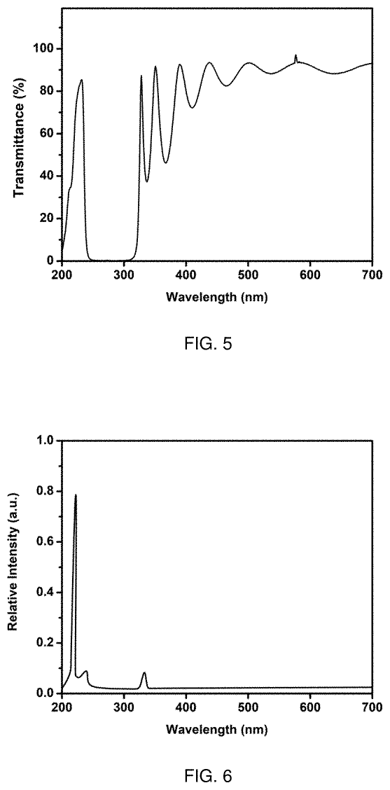

FIG. 2 is an embodiment of the multi-band germicidal irradiation apparatus of the present disclosure 100. The apparatus 100 includes an excimer lamp 101, a driver 102, a controller 103, and a band-stop filter 104. The driver 102 converts an external power to an internal power for activating the excimer lamp 101. The excimer lamp has four mini tubes as shown in FIG. 3. In one embodiment, the four mini tubes are filled with a mixture of KrCl and Kr₂Cl at 600 mbar. This excimer lamp has a main peak wavelength at 222 nm due to KrCl and a small peak wavelength at 325 nm due to Kr₂Cl as shown in FIG. 4. The transmittance of a band-stop filter is shown in FIG. 5. This band-stop filter 104 filters effectively the wavelengths in the 240˜315 nm range. FIG. 6 shows the result of applying the band-stop filter 104 to the excimer lamp 101 with KrCl and Kr₂Cl. The wavelengths in the 200˜230 nm range are passed through, the wavelengths in the 240˜315 nm range are substantially reduced, and there is still residual wavelengths (about 0.02) in the visible wavelength range.

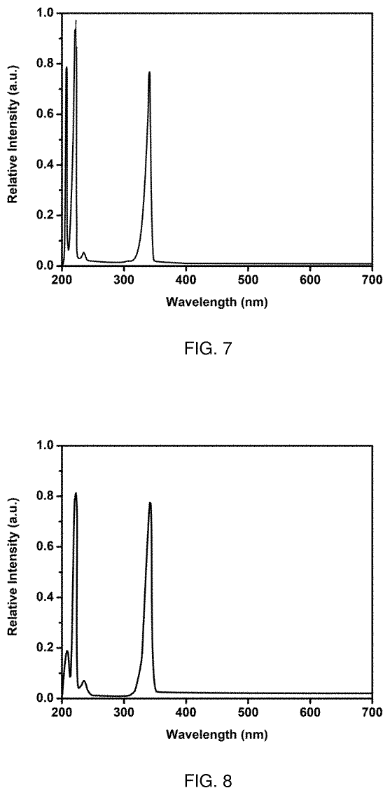

In another embodiment, the four mini tubes of the excimer lamp shown in FIG. 3 are filled with two different gases: mini tubes #1 and #3 are filled with KrCl, whereas mini tubes #2 and #4 are filled with I₂, both at 200 mbar. The spectral power distribution of this excimer lamp is shown in FIG. 7. This excimer lamp has a peak wavelength at 222 nm due to KrCl and another peak wavelength at 342 nm wavelength due to I₂. FIG. 8 shows the result of applying the band-stop filter 104 to the excimer lamp with KrCl and I₂. Again, wavelengths in the 200˜230 nm range are passed through and the wavelengths in the 240˜315 nm range are substantially reduced. More importantly, the UVA wavelengths in the 315 nm˜400 nm range are preserved, especially the peak wavelength at 342 nm, together with the residual visible wavelengths.

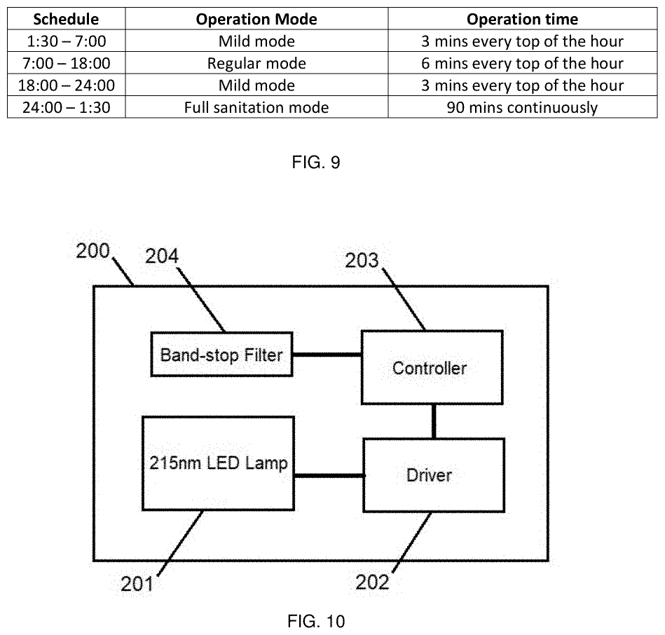

Though not shown, the controller 103 has a fixed, built-in schedule for operating the excimer lamp 101 as shown in FIG. 9. From 1:30-24:00, the controller would operate the excimer lamp intermittently, and the ON time would depend on the office hours. During the office hours (7:00-18:00), the controller will operate the excimer lamp at a regular mode by turning on the excimer lamp for six minutes on top of the hour. The six minutes per hour operation is calculated such that when extrapolating over an eight-hour period, any occupant in the environment will not receive any UV dosage exceeding the UV TLV dosage defined by ACGIH. During 1:30-7:00 and 18:00-24:00, since there would be lesser occupants in the environment, the controller will operate the excimer lamp at a mild mode by turning on the excimer lamp for three minutes on top of the hour. The three minutes per hour operation is calculated such that when extrapolating over an eight-hour period, the UV dosage received by any occupant in the environment object is at least 25% below the UV TLV dosage defined by the ACGIH. During 24:00-1:30, the controller will operate the excimer lamp at a full sanitation mode by turning on the excimer lamp continuously for ninety minutes to disinfect the environment thoroughly.

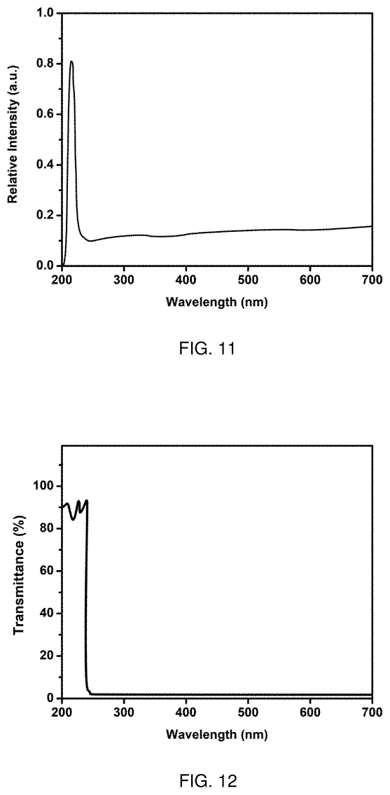

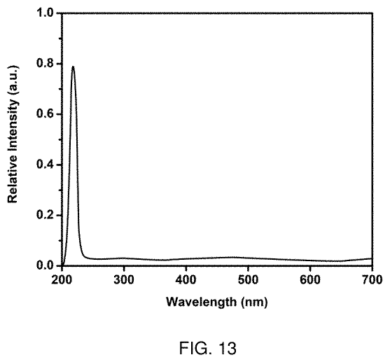

FIG. 10 is another embodiment of the multi-band germicidal irradiation apparatus of the present disclosure 200. The apparatus 200 includes an LED lamp 201, a dimmable driver 202, a controller 203, and a band-stop filter 204. The driver 202 converts an external power to an internal power for activating the LED lamp 201. The LED lamp 201 uses aluminum nitride LED and has one peak wavelength at 215nm. Its spectral power distribution is shown in FIG. 11. The transmittance of the band-stop filter 204 is shown in FIG. 12. This filter would significantly reduce the bandwidths in the 240˜400 nm range. FIG. 13 shows the spectral power distribution of the LED lamp 201 after being filtered by the band-stop filter 204. The bandwidths in the 240˜400 nm range is indeed significantly reduced, while there is still residual wavelength (around 0.01) in the 400˜700 nm range. The controller 203 will operate the LED lamp 201 according to a similar schedule as shown in FIG. 9, but with one significant difference, that is, the controller 203 will operate the LED lamp 201 continuously, not intermittently. The controller 203 is configured to operate different operation modes (regular, mild, full sanitation) by adjusting the radiant power emitted by the LED lamp 201 through tuning the dimmable driver 202.

### ADDITIONAL AND ALTERNATIVE IMPLEMENTATION NOTES

Although the techniques have been described in language specific to certain applications, it is to be understood that the appended claims are not necessarily limited to the specific features or applications described herein. Rather, the specific features and examples are disclosed as non-limiting exemplary forms of implementing such techniques. As used in this application, the term “or” is intended to mean an inclusive “or” rather than an exclusive “or.” That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more,” unless specified otherwise or clear from context to be directed to a singular form.

What is claimed is:

1. A multi-band germicidal irradiation apparatus, comprising:
a radiation source;
a driver; and
an optical filter,
wherein:
the driver is configured to convert an external power to an internal power to activate the radiation source,
the radiation source is configured to generate a first wavelength in a wavelength range of 190˜230 nm and a second wavelength in a wavelength range of 315˜700 nm,
the optical filter is configured to reduce over 50% of a radiation of the radiation source in a wavelength range of 240˜315 nm, and
the optical filter is configured to transmit at least a part of the radiation of the radiation source in the wavelength ranges of 190˜230 nm and 315˜700 nm.

2. The apparatus of claim 1, wherein the radiation source comprises an excimer lamp having a gas or a combination of a plurality of gases to produce the first wavelength in the wavelength range of 190˜230 nm and the second wavelength in the wavelength range of 315˜700 nm.

3. The apparatus of claim 2, wherein the excimer lamp comprises krypton-chloride (KrCl), krypton-bromine (KrBr), argon-fluorine (ArF), krypton-iodine (KrI), or a combination thereof, and wherein the excimer lamp is configured to generate the first wavelength in the wavelength range of 190˜230 nm.

4. The apparatus of claim 2, wherein the excimer lamp comprises iodine (I₂), xenon-fluorine (XeF), or a combination thereof, and wherein the excimer lamp is configured to generate a wavelength in a wavelength range of 315˜400 nm.

5. The apparatus of claim 2, wherein the excimer lamp comprises a gas configured to generate a wavelength in a wavelength range of 400˜700 nm.

6. The apparatus of claim 2, wherein the excimer lamp comprises two or more gases, and wherein each of the two or more gases is stored in a separate enclosed chamber and the two or more gases are not mixed.

7. The apparatus of claim 1, wherein the radiation source comprises one or more light emitting diodes (LEDs) configured to emit the first wavelength in the wavelength range of 190˜230 nm and the second wavelength in the wavelength range of 315˜700 nm.

8. The apparatus of claim 7, wherein the radiation source comprises more than one types of LED, and wherein each type of LED has a different spectral power distribution.

9. The apparatus of claim 1, further comprising a controller, wherein the controller is connected to the driver and is configured to adjust a radiant power emitted by the radiant source, and wherein the controller is configured to limit a total dispensed ultraviolet (UV) dosage (TDD) emitted by the apparatus over a predefined period not to exceed a UV threshold limit value (TLV) dosage defined by American Conference of Governmental Industrial Hygienists (ACGIH).

10. The apparatus of claim 9, wherein the TDD is controlled by either or both of:
the controller adjusting an operating time of the radiant source during the predefined period, and the controller adjusting the radiant power emitted by the radiant source.

11. A multi-band germicidal irradiation apparatus, comprising:
a radiation source;
a driver;
a controller; and
an optical filter,
wherein:
the driver is configured to convert an external power to an internal power to activate the radiation source,
the controller is connected to the driver and is configured to adjust a radiant power emitted by the radiant source,
the radiation source is configured to generate a first wavelength in a wavelength range of 190˜230 nm and a second wavelength in a wavelength range of 400˜700 nm,
the optical filter is configured to reduce over 50% of a radiation of the radiation source in a wavelength range of 240˜400 nm,
the optical filter is configured to transmit at least a part of the radiation of the radiation source in the wavelength ranges of 190˜230 nm and 400˜700 nm,
the controller is configured to limit a total dispensed UV dosage (TDD) emitted by the apparatus over a predefined period not to exceed a threshold limit value (TLV) dosage defined by American Conference of Governmental Industrial Hygienists (ACGIH), and
the TDD is controlled by either or both of:
the controller adjusting an operating time of the radiant source during the predefined period, and
the controller adjusting the radiant power emitted by the radiant source.

12. The apparatus of claim 11, wherein the apparatus supports a mild mode operation, and wherein, when operating in the mild mode, the controller is configured to operate the radiant source such that a UV dosage received by an object is at least 25% below the TLV dosage defined by the ACGIH.

13. The apparatus of claim 11, wherein the apparatus supports a boost mode operation, and wherein, when operating in the boost mode, the controller is configured to operate the radiant source such that a UV dosage received by an object is at least 25% above the TLV dosage defined by the ACGIH.

14. The apparatus of claim 11, wherein the apparatus supports a full sanitation mode operation, and wherein, when operating in the full sanitation mode, the controller is configured to maximize the radiant power emitted by the radiant source over less than 4 hours.
